(19) United States
(12) Patent Application Publication
(10) Pub. No.: US 2023/0052622 A1
(43) Pub. Date: Feb. 16, 2023

(54) RECEIVER COMPRISING COILS FOR WIRELESSLY RECEIVING POWER

(71) Applicant: Creo Medical Limited, Chepstow (GB)

(72) Inventors: Christopher Paul Hancock, Chepstow (GB); Edward Waller, Chepstow (GB); Shaun Preston, Chepstow (GB)

(73) Assignee: Creo Medical Limited, Chepstow (GB)

(21) Appl. No.: 17/790,112

(22) PCT Filed: Dec. 17, 2020
(86) PCT No.: PCT/EP2020/086803

§ 371 Date: Jun. 29, 2022

(30) Foreign Application Priority Data
Jan. 6, 2020 (GB) 2000115.2

(51) Int. Cl. A61B 1/00; A61B 1/04; A61B 18/18; H02J 50/12; H02J 50/40; H02J 50/00

(52) U.S. Cl. CPC A61B 1/00029; A61B 1/041; A61B 18/1815; H02J 50/12; H02J 50/402; H02J 50/005; A61B 2018/1823; A61B 2018/00494

(57) ABSTRACT

A receiver (6) is disclosed for wirelessly receiving power from a transmitter. The receiver comprises a resonant receiver circuit having a plurality of coils (200a)-(200d) operatively coupled to a combining circuit (202). Each coil, with the combining circuit, is arranged to receive power via resonant inductive coupling. The combining circuit is arranged to combine power received from the plurality of coils for provision to an electric load. Other embodiments provide a capsule for ingestion by a patient, the capsule comprising the receiver.

### FIELD OF THE INVENTION

The invention relates to a receiver for wirelessly receiving power from a transmitter, a capsule for ingestion by a patient, and a wireless power transfer system comprising a transmitter and either the receiver or the capsule. Specific embodiments involve wireless power transfer via resonant inductive coupling, wherein the receiver is adapted to receive power from the transmitter when the receiver is orientated at multiple different angles with respect to the transmitter and/or when the receiver is positioned a multiple different distances from the transmitter. Other embodiments relate to a capsule containing the receiver, wherein the capsule is to be ingested by a patient for medical purposes. For example, the capsule may include an electrosurgical device for delivering high-frequency energy to a treatment site inside the patient for tissue treatment, such as, coagulation or ablation.

### BACKGROUND TO THE INVENTION

Capsule endoscopy is a procedure that uses a tiny wireless camera to take pictures of a patient's gastrointestinal tract. A capsule endoscopy camera sits inside a vitamin-size capsule that a patient swallows. As the capsule travels through the patient's gastrointestinal tract, the camera takes multiple pictures that are transmitted to a recorder the patient wears on a belt around their waist. Capsule endoscopy helps doctors see inside the patient's small intestine—an area that isn't easily reached with more-traditional endoscopy procedures. Traditional endoscopy involves passing a long, flexible tube equipped with a video camera down the patient's throat or through their rectum. The capsule may include a battery which powers any on-board electronics.

Inductive power coupling allows energy to be transferred from a power supply to an electric load without there being a wired connection between the power supply and the electric load. Specifically, a power supply is wired to a primary coil and an oscillating current signal is sent through the primary coil which induces an oscillating magnetic field around the primary coil. The oscillating magnetic field induces an oscillating voltage signal in a secondary coil, placed close to the primary coil. In this way, electrical energy may be transmitted from the primary coil to the secondary coil by electromagnetic induction without the two coils being conductively connected.

When electrical energy is transferred from a primary coil to a secondary coil, the inductors are said to be inductively coupled. An electric load wired in series with such a secondary coil may draw energy from the power source wired to the primary coil when the secondary coil is inductively coupled thereto.

Non-resonant coupled inductors, such as typical transformers, work on the principle of a primary coil generating a magnetic field and a secondary coil subtending as much as possible of that field so that the power passing through the secondary coil is as close as possible to that of the primary coil. This requirement that the field be covered by the secondary coil results in very short range and usually requires a magnetic core. Over greater distances the non-resonant induction method can waste a majority of the energy in resistive losses of the primary coil, and so be inefficient.

Using resonance can dramatically improve efficiency. If resonant coupling is used, the secondary coil is capacitive loaded so as to form a tuned inductor-capacitor (LC) circuit. If the primary coil is driven at the secondary side resonant frequency, significant power may be transmitted between the coils over a range of a few times the coil diameters at reasonable efficiency. That is, the strength of the induced voltage in the secondary coil varies according to the oscillating frequency of the electrical current sent through the primary coil, and the induced voltage is strongest when the oscillating frequency equals the resonant frequency of the system. The resonant frequency depends upon the inductance and the capacitance of the system.

It is further noted that known inductive power transfer systems typically transmit power at the resonant frequency of the inductive couple. This can be difficult to maintain as the resonant frequency of the system may fluctuate during power transmission, for example in response to variations in alignment between primary and secondary coils, and variations in distance between the primary and secondary coils.

A system for inductive power transfer between a resonant transmitter circuit and a resonant receiver circuit is described in “Lee, S.-H., 2011. A Design Methodology for Multi-kW, Large Airgap, IEEE”. Specifically, a system is provided for high power transfer which may be suitable for charging smart phones or electric cars, and in other scenarios in which the transmitter and receiver are in a fixed distance and orientation with respect to each other during the power transfer.

There is a need for an inductive power transfer system with a higher tolerance to variations in inductive coil alignment and spacing. The present invention addresses this need.

### SUMMARY OF THE INVENTION

At its most general, the present invention provides a wireless power transfer system including a transmitter which transmits power to a receiver via resonant inductive coupling. Additionally, the receiver includes a plurality of secondary coils, each of which are configured to receive power from a primary coil in the transmitter via resonant inductive coupling. The power received by different ones of the plurality of secondary coils can be combined together to form a combined power signal, which can be used to power an electric load in or attached to the receiver.

Additionally, different ones of the secondary coils can be orientated at different angles to each other meaning that they are configured for optimal power transfer with the transmitter at different orientation angles between the transmitter and receiver. In this way, when the receiver is at an orientation angle with the transmitter in which one of secondary coils cannot receive power from the primary coil, another one of the secondary coils can be in an orientation in which it can receive power from the transmitter. Additionally, when the receiver is at an orientation angle with the transmitter in which multiple secondary coils can only receive power sub-optimally, the received power from these multiple secondary coils can be combined together. In this way, power can be passed from the transmitter to the receiver regardless of an orientation angle between the transmitter and receiver. This received power can be used to power an electric load of the receiver.

Further, different ones of the secondary coils can be configured for critical coupling (i.e. optimal power transfer) at different distances between the transmitter and receiver. In this way, when the receiver is spaced from the transmitter by a distance that is too close or too far away for one of the secondary coils to receive power from the primary coil, another one of the secondary coils can be spaced from the transmitter by a distance at which it can receive power from the transmitter. Additionally, when the receiver is spaced from the transmitter by a distance at which multiple secondary coils can only receive power sub-optimally, the received power from these multiple secondary coils can be combined together. In this way, power can be passed from the transmitter to the receiver at a wider range of distances between the transmitter and receiver than would be achievable with fewer secondary coils. This received power can be used to power an electric load of the receiver.

Furthermore, the receiver may be contained within a capsule (e.g. a pill-shaped capsule) for insertion into (e.g. ingestion by) a patient for medical purposes. The capsule may include electronics for enabling or assisting various medical procedures, and this electronics may be powered by power passed from the transmitter to the receiver via resonant inductive coupling. For example, the electronics may include an electrosurgical device for generating and delivering high-frequency energy (e.g. radio frequency (RF) electromagnetic (EM) energy and/or microwave EM energy). In this way, the capsule may travel to a treatment site inside the patient's body and, once at the treatment site, deliver the high-frequency energy to biological tissue at the treatment site. The delivered energy may be used to ablate or coagulate biological tissue at the treatment site.

According to a first aspect of the invention, there is provided a receiver for wirelessly receiving power from a transmitter, the receiver comprising a resonant receiver circuit having a plurality of coils operatively coupled to a combining circuit, wherein each coil, with the combining circuit, is arranged to receive power via resonant inductive coupling, and wherein the combining circuit is arranged to combine power received from the plurality of coils for provision to an electric load. In this way, multiple coils within the receiver can be used to receive power from a transmitter via resonant inductive coupling. Also, the power received by individual ones of the plurality of coils can be combined together into a combined power signal for powering an electric load which may be part of the receiver or may be a separate element or module which is electrically coupled to the receiver.

At least two coils of the plurality of coils may be orientated at different angles to each other. In an embodiment, each of the coils may be orientated at a different angle to each other coil, i.e. each coil may be orientated at a unique angle. For example, the coils may be arranged around the circumference of a roughly oval or circular shape. The coils may uniformly circumferentially spaced, i.e. spaced at regular intervals around the circumference. Additionally or alternatively, the coils may be non-uniformly circumferentially spaced, i.e. spaced at irregular intervals around the circumference. It is to be understood however that the coils may be arranged in any shape, for example, a square, a triangle, a rectangle, or an irregular shape.

Also, at least two coils of the plurality of coils are configured for critical coupling at different distances to each other, i.e. different spacings from the transmitter's primary coil. In an embodiment, each of the coils may be configured for critical coupling at a different distance to each other, i.e. at a different spacing from the transmitter's primary coil. The expression “configured for critical coupling at a distance X” is understood to mean that the coil is physically formed or structured so that when a spacing between this coil and the transmitter coil is within a specific distance range X (aka the “critical zone”), power transfer is optimal or most efficient (e.g. optimal efficiency may be 50% to 95%). For example, inside the critical zone, power transfer may be 70% efficient but, outside the critical zone, power transfer may be less than 70% efficient. Generally, as the spacing between the transmitter and receiver coils moves away from the critical zone, the maximum achievable power transfer decreases exponentially. Hence, for the coil in question, distance range X represents the range of distances at which power transfer is optimal or maximally efficient. The physical or structural variables of the coil which influence the distance range (i.e. the critical zone) within which critical coupling occurs include: a coil inductance, a number of turns in the coil, a permeability of the material (e.g. wire) used to form the coil, a cross-sectional area of the material (e.g. wire) used to form the coil, a length of the coil, and skin effect of the material (e.g. wire) used to form the coil. It is noted that there are two terms often used to define the relationship between a primary transmitter coil and a secondary receiver coil: coupling coefficient and coupling strength. The coupling coefficient relates to a ratio of the power transmitted from the primary coil vs. the power received by the secondary coil, multiplied by the transformer ratio for the specific primary and secondary coils. The transformer ratio is a ratio between the current and voltage of the primary coil and the current and voltage of the secondary coil. The coupling strength relates to the efficiency of power transferred between the primary coil and secondary coil in relation to the physical characteristics of the coils and the distance between them. As such, coupling strength is a maximum in the critical zone, which is the range of distances in which critical coupling occurs.

The combining circuit may include a plurality of impedance (e.g. capacitive) elements. Each impedance element may be microstrip transmission line. Each impedance element may be a quarter wave transformer, which can be fabricated as a quarter wave line on a printed circuit board, a coaxial cable, or a lumped circuit element (e.g. capacitor). In any case, the plurality of impedance elements are connected together to form a circuit which combines power received from each of the plurality of coils into a combined power signal that is provided to an output of the combining circuit. Also, each coil is coupled to the output of the combining circuit by a combination of impedance elements, and that combination of impedance elements has a characteristic impedance (e.g. capacitive impedance) which combines together with that coil to form a resonant circuit for receiving power via resonant inductive coupling. In an embodiment, a combination of impedance elements which couple one of the coils to the output is different to a combination of impedance elements which connect a different one of the coils to the output. In a further embodiment, each coil is coupled to the output of the combining circuit via a unique combination of impedance elements.

The combining circuit may include a plurality of power combiners. Each power combiner may have two input ports coupled to an output port, and each power combiner may be operable to provide at its output port a combination of separate power signals received at both input ports. Also, the plurality of power combiners are connected together to combine power received from each of the plurality of coils into a combined power signal that is provided to an output of the combining circuit. Further, each power combiner may have a characteristic impedance, for example, each power combiner may act as a lumped element having a particular characteristic impedance. That is, each power combiner may form a signal adder based on lumped elements, e.g. a combination of series inductors and shunt capacitors. Furthermore, each coil may be coupled to the output of the combining circuit by a combination of power combiners having characteristic impedances which combine together with that coil to form a resonant circuit for receiving power via resonant inductive coupling. Therefore, the combining circuit performs two functions. Firstly, the combining circuit forms a plurality of capacitive circuits, wherein each of these capacitive circuits connects to one of the coils to form a resonant circuit for receiving power from the transmitter via resonant inductive coupling. That is, the coil provides the L and the capacitive circuit provides the C which combine together to form a resonant LC circuit. In an embodiment, the number of capacitive circuits matches the number of coils in the plurality of coils, and each capacitive circuit is associated with (e.g. connected to) a different one of the coils. Secondly, the combining circuit combines together the power received via each coil into a combined power signal which can be used to power an electric load that is either part of or electrically coupled to the receiver.

Considering the combining circuit in more detail, the plurality of power combiners may be grouped into multiple stages including a first stage (e.g. stage 1). The number of first stage power combiners may match the number of coils in the plurality of coils. Also, each first stage power combiner may be associated with (e.g. connected to) a different coil of the plurality of coils. Further, each first stage power combiner may have a first input port connected to a first end of its associated coil, and a second input port connected to a second end of its associated coil.

Additionally, the multiple stages may include one or more further stages. For each further stage:

the number of power combiners in that further stage (e.g. stage 2) may match half the number of power combiners in an adjacent previous stage (e.g. stage 1);

each power combiner in that further stage may be associated with (e.g. connected to) a different pair of power combiners from the adjacent previous stage;

each power combiner from the adjacent previous stage may only be associated with (e.g. connected to) a single power combiner from that further stage;

each power combiner in that further stage may have: (i) a first input port connected to the output port of one of its associated pair of power combiners from the adjacent previous stage; and, (ii) a second input port connected to the output port of the other of its associated pair of power combiners from the adjacent previous stage.

Additionally, the multiple stages may include a final stage. The final stage may include a single power combiner. That is, one or more further stages may be provided in-between the first stage and the final stage so that the number of power combiners in a stage are reduced from the number of coils (i.e. the first stage) to one (i.e. the final stage). That is, after the first stage, a chain or series of further stages may be added, to half the number of power combiners in a stage until a further stage is formed having only two power combiners, at which point a final stage is added to finish the chain or series. For example, if there are eight coils, then the first stage will include eight power combiners. In this case, a further two stages are required to reduce the number of power combiners in a stage to two, i.e. a first further stage having four power combiners, followed by a second further stage having two power combiners. In any case, the single power combiner of the final stage may be associated with (e.g. connected to) a pair of power combiners from the adjacent previous stage (e.g. the only two power combiners in the adjacent previous stage). Specifically, the single power combiner of the final stage has: (i) a first input port connected to the output port of one of the pair of power combiners from the adjacent previous stage; and, (ii) a second input port connected to the output port of the other of the pair of power combiners from the adjacent previous stage. Additionally, the final stage may include an output impedance element coupled in-between the output of the single power combiner and the output of the combining circuit.

It is to be understood that the combining circuit may have any number of stages, but that the number of stages depends on the number of coils in the plurality of coils. For example, where the number of coils (N) is a square of two, the number of power combiners in the combining circuit will be 2N−1, and the number of stages will be 2 to the power of (N−1) or 2^{(N-1)}. However, it is to be understood that the plurality of coils may include any number of coils, and so the combining circuit may include any number of power combiners, and any number of stages.

Furthermore, connections between the plurality of power combiners in the combining circuit are selected (e.g. set, fixed or established) to minimise differences between the power signals provided at the first and second input ports of each power combiner. In this way, balance in the combining circuit is improved which, in turn, improves the way in which the combining circuit fulfils it two objectives ((i) to form a resonant circuit with each coil to receive power via resonant inductive coupling, and (ii) to combine together the power received via each coil into a combined power signal for provision to an electric load). More specifically, the amount of power received via each receiver coil is unpredictable because, for example, the amount of power is dependent on an orientation or separation distance between each receiver coil and the transmitter coil. Therefore, it is difficult to guarantee that the combining circuit is balanced. Stated differently, it is difficult to guarantee that the amount of power received by a first input port of a given power combiner is the same as the amount of power received by a second input port of that power combiner. Also it is difficult to ensure that the frequency of the signal received by the first input port cooperates with the frequency of the signal received by the second input port, wherein frequencies “cooperate” if interference (constructive or destructive) is avoided or reduced. Therefore, in an attempt to improve balance, power combiners and groups of power combiners may be paired together based on their received average power to improve balance in the combining circuit, and thereby to improve power transfer to the receiver and power delivery to the electric load. For example, in a further stage (e.g. stage 2) the power combiners from an adjacent previous stage (e.g. stage 1) are paired together based their average power output. More specifically, the pairing together may be based on their average power output to an example or test scenario, for example, the transmitter and receiver may be held in a fixed relationship and the transmitter used to transmit a test power signal. Then the average power received at each receiver coil may be measured. The coils may then be grouped into pairs which received the most similar average power. Further, each stage of power combiners may be connected by pairing together average power signals from the previous stage that are most similar to each other. Additionally, one or more stages may include directional diodes or filters at specific frequencies to minimise constructive and/or destructive interference, for example, to maintain an overall combined direction of current flow.

It is to be understood that whilst embodiments may include the use of power combiners having two input ports and a single output port, in at least some other embodiments, different power combiner structures may be used. For example, each power combiner may have more than two input ports, for example, three, four, five or more. In any case, each power combiner functions to combine together the signals received on each input port into a combined signal which is output from the output port of the power combiner. In this case, as in the embodiments explained above, the plurality of power combiners are connected together to combine power received from each of the plurality of coils into a combined power signal that is provided to an output of the combining circuit. Also, each coil is coupled to the output of the combining circuit by a combination of power combiners (or impedance elements), and this combination of power combiners (or impedance elements) has a characteristic impedance (e.g. capacitive impedance) which combines together with that coil to form a resonant circuit for receiving power via resonant inductive coupling. As above, each power combiner may have a characteristic impedance, for example, each power combiner may act as a lumped element having a particular characteristic impedance. That is, each power combiner may form a signal adder based on lumped elements, e.g. a combination of series inductors and shunt capacitors.

In an embodiment, at least one of the power combiners is a Wilkinson power combiner, for example, each of the power combiners may be a Wilkinson power combiner. Additionally, in an embodiment, at least one of the power combiners is formed from a microstrip electrical transmission line, for example, each of the power combiners may be formed from a microstrip electrical transmission line.

In an embodiment, the receiver includes an electric load (or electric circuit, device, apparatus or appliance) coupled to the combining circuit to receive power therefrom. In this way, the power received by the receiver via resonant inductive coupling may be used to power the electric load. For example, the electric load may be arranged to convert the power into some other form, such as, thermal energy, sound energy, electromagnetic energy (e.g. RF and/or microwave energy). For example, the electric load may include a rectifier to convert the alternating or oscillating power received from the combining circuit into a direct current (DC) signal. Also, the electric load may include an electrosurgical apparatus or device for generating and delivering high-frequency electromagnetic energy (e.g. RF and/or microwave energy) into a treatment site around (e.g. surrounding) the receiver for treating biological tissue in the treatment site. More specifically, the electrosurgical apparatus may include: a microwave power amplifier coupled to the rectifier for generating microwave electromagnetic energy from the DC signal produced by the rectifier; and, a transmission line coupled to the microwave power amplifier for delivering the microwave electromagnetic energy into biological tissue in the treatment site. Also, the transmission line may be arranged to have an impedance that matches an impedance of a target biological tissue in the treatment site. For example, the electrosurgical device may be intended for use to treat a particular type of biological tissue (aka target tissue type), having a particular characteristic impedance. In this case, the transmission line may be structurally arranged to have a characteristic impedance which substantially matches the characteristic impedance of the target tissue type. For instance, a capacitance or resistance of the transmission line may be set in order that the characteristic impedance of the transmission line matches that of the target tissue type.

The electric load may include a sensor for generating an electrical signal based on (e.g. representing) an environment (e.g. the physical surroundings) of the receiver. The sensor is powered by the DC signal generated by the aforementioned rectifier of the electric load. Also, the sensor is coupled to the combining circuit such that the sensor provides its electrical signal to the output of the combining circuit. In an embodiment, the sensor may be an imaging module which captures an image of part of the environment which surrounds the receiver (e.g. the part which faces the imaging module). The image may be captured via visible light, infrared light, or ultraviolet light. As such, the electrical signal represents an image and, thereby represents an environment of the receiver. The electrical signal may define a simple binary image (e.g. black and white), or a more complex image (e.g. greyscale or colour). Furthermore, the receiver may operate as a transmitter for transmitting sensor data via resonant inductive coupling. Specifically, each coil, with the combining circuit, provides a resonant transmitter circuit arranged to transmit the electrical signal via resonant inductive coupling. That is, the same resonant circuit used to receive power signals from the transmitter is used to transmit sensor data to the transmitter. Accordingly, whilst the combining circuit acts as a combining circuit for power signals, the combining circuit may also act as a splitting circuit for sensor signals. In an embodiment, the electric load further includes a signal conditioning unit operatively coupled in-between the sensor and the combining circuit. The signal conditioning unit may be coupled to the rectifier so as to be powered by its DC signal. The signal conditioning unit operates to vary a characteristic of the electrical signal before the signal is transmitted via resonant inductive coupling. The characteristic may be a magnitude, voltage or frequency of the electrical signal. For example, it may be necessary to amplify a magnitude (or voltage) of the electrical signal so that it has sufficient power to be transmitted via resonant inductive coupling and received by the transmitter. As such, the signal conditioning unit may include a power amplifier to amplify the electrical signal from the sensor before the signal is provided to the combining circuit. Additionally, it may be necessary to change a frequency of the electrical signal to avoid or reduce any interference (e.g. constructive or destructive) between the electrical signal transmitted from the receiver and the power signal received by the receiver. This is necessary because the transmission path and the reception path include both the coils and the combining circuit. As such, the signal conditioning unit may include a frequency divider (e.g. to reduce frequency) and/or a frequency multiplier (e.g. to increase frequency). As such, the signal conditioning unit conditions the sensor output so that it is suitable for transmission via resonant inductive coupling and to reduce/avoid interference with the power signal. In an embodiment, the signal conditioning unit conditions the power level of the electrical signal transmitted from the receiver to be less than the power level of the power signal received by the receiver. In summary, whilst the receiver is configured to operate as a receiver for receiving power via resonant inductive coupling, the receiver may also be configured to operate as a transmitter for transmitting data (e.g. sensor or image data) via resonant inductive coupling. It is also envisioned that the receiver may transmit data or information regarding the efficiency of the power transfer between the transmitter and the receiver.

According to a second aspect of the invention, there is provided a capsule (or device) for ingestion by (or insertion into) a patient, the capsule comprising a housing containing a receiver according to the first aspect. In an embodiment, a shape of the housing is substantially sphero-cylindrical (or pill-shaped, i.e. shaped like a pharmaceutical pill).

In this way, the receiver may be ingested or inserted into a patient, for example, swallowed by the patient in order to enter the patient's gastrointestinal tract. The capsule may receive power via resonant inductive coupling, as described above. Moreover, the above-described first aspect provides a mechanism for delivering power via resonant inductive coupling wherein power transfer can be insensitive to an orientation between the transmitter and receiver. This is particularly advantageous in the context of an ingestible or insertable capsule since, once the capsule enters the patient's body, it can be hard to fix an orientation of the capsule relative to a transmitter outside the patient's body. Moreover, the above-described first aspect provides a mechanism for delivering power via resonant inductive coupling wherein power transfer can be achieved across a wide range of separation between the transmitter and receiver. Again, this is particularly advantageous in the context of an ingestible or insertable capsule since, once the capsule enters the patient's body, it can be hard to fix a separation distance between the capsule and a transmitter outside the patient's body.

In an embodiment, the capsule housing may be formed from a biocompatible material (e.g. Parylene C or PTFE). Alternatively, a layer of biocompatible material may be applied to an outer surface of the housing. The biocompatible layer may have a thickness of 10 μm or less.

In an embodiment, the coils may be arranged around or close to an inside edge of the capsule housing. For example, when the capsule housing is sphero-cylindrical or pill-shaped, the coils may be arranged in a roughly oval shape which generally follows an inside surface of the housing. That is, an outside surface of the oval shape may be substantially opposite and adjacent to an inside surface of the housing. In this way, the coils may be spread out in the capsule to improve power transfer.

In an embodiment, the capsule may have an electric load (aka powered circuitry) including the above described electrosurgical device of the first aspect. Accordingly, the capsule may be passively or actively transported to a treatment site inside the patient's body and, once at the treatment site, the capsule may generate and deliver high-frequency EM energy (e.g. RF and/or microwave) to biological tissue surrounding the capsule. This energy may be used to coagulate and/or ablate the biological tissue as part of a medical procedure. In an embodiment, a magnetic steering apparatus may be used to guide the capsule to the treatment site via magnetic attraction and/or magnetic repulsion. For example, the capsule may include a magnetic or ferrous portion which reacts with the magnetic steering apparatus that is located outside the patient's body.

Additionally, the capsule's electric load may further include an imaging device (e.g. a camera) for inspecting and monitoring internal structures (e.g. vascular structures) of a patient. For example, the imaging device may be configured to capture images in timed intervals (e.g. twice per second). In this case the housing of the capsule may include a window portion which is substantially transparent such that the imaging device can see through the housing to capture images. Also, the electric load may include a light source to illuminate the tissue surrounding the window each time the imaging device captures a new image. Additionally or alternatively, the electric load may include one or more biosensors for detecting the presence or concentration of a biological analyte, such as a biomolecule, a biological structure or a microorganism. In this case, the housing of the capsule may have an aperture which allows at least part of the biosensor to contact tissue in treatment site surrounding the capsule. Additionally or alternatively, the electric load may include a thermal module (e.g. heating element) for changing the temperature of tissue at the treatment site. For example, the thermal module may be used to heat tissue (e.g. cancerous tissue) at the treatment site to activate heat activated drugs, such as, heat activated chemotherapy drugs.

In a third aspect of the present invention, there is provided a wireless power transfer system comprising: a transmitter for wirelessly transmitting power, the transmitter comprising a resonant transmitter circuit having a coil arranged to transmit power wirelessly via resonant inductive coupling, and a receiver according to the first aspect, or a capsule according to the second aspect, for wirelessly receiving power from the transmitter.

In an embodiment, the transmitter may include a power signal source electrically coupled with a primary inductive coupler (or transmitter antenna or coil). The power signal source provides an oscillating current signal to the primary inductive coupler, and the primary inductive coupler generates via electromagnetic induction an oscillating magnetic field from the oscillating current signal. The oscillating magnetic field provides a mechanism for wirelessly transferring power from the transmitter to the receiver. In this way, the transmitter need not be electrically coupled to the receiver.

Herein, radiofrequency (RF) may mean a stable fixed frequency in the range 10 kHz to 300 MHz and microwave frequency may mean a stable fixed frequency in the range 300 MHz to 100 GHz. Preferred spot frequencies for the RF energy include any one or more of: 100 kHz, 250 kHz, 400kHz, 500 kHz, 1 MHz, 5 MHz. Preferred spot frequencies for the microwave energy include 915 MHz, 2.45 GHz, 5.8 GHz, 14.5 GHz, 24 GHz.

The term “electrosurgical” is used in relation an instrument, apparatus or tool which is used during surgery and which utilises microwave and/or radiofrequency electromagnetic (EM) energy.

### BRIEF DESCRIPTION OF THE DRAWINGS

Embodiments of the invention are discussed below with reference to the accompanying drawings, wherein like reference signs relate to like features and, in which:

FIG. 1 is a schematic diagram of a wireless power transfer system, in accordance with an embodiment;

FIG. 2 is a circuit diagram of a transmitter of the wireless power transfer system of FIG. 1, in accordance with an embodiment;

FIG. 3 is a circuit diagram of a receiver of the wireless power transfer system of FIG. 1, in accordance with an embodiment;

FIG. 4 is a schematic diagram illustrating the relative orientations of the coils of the receiver of FIG. 3, in accordance with an embodiment;

FIG. 5 is a schematic diagram illustrating the relative orientations of the coils of a receiver of the wireless power transfer system of FIG. 1, in accordance with a further embodiment;

FIG. 6 is a circuit diagram of the receiver of FIG. 5, in accordance with an embodiment;

FIG. 7 is a circuit diagram of powered circuity, in accordance with an embodiment;

FIG. 8 is a schematic diagram of a capsule for ingestion by a patient for medical purposes, in accordance with an embodiment; and

FIG. 9 is a schematic diagram of a capsule for ingestion by a patient for medical purposes, in accordance with another embodiment.

### DETAILED DESCRIPTION; FURTHER OPTIONS AND PREFERENCES

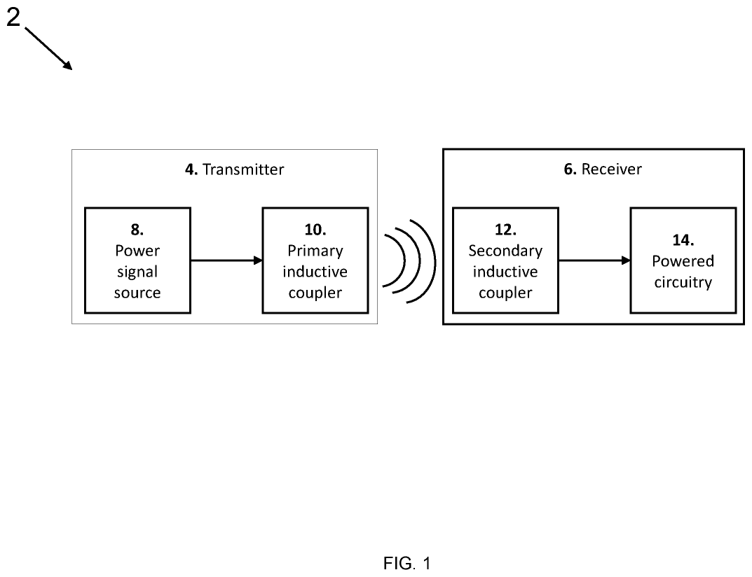

FIG. 1 illustrates a wireless power transfer system 2 comprising a transmitter 4 and a receiver 6. In operation, the transmitter 4 wirelessly transmits power to the receiver 6 via resonant inductive coupling. Specifically, the transmitter 4 includes a power signal source 8 electrically coupled with a primary inductive coupler (or transmitter antenna) 10. The power signal source 8 provides an oscillating current signal to the primary inductive coupler 10, and the primary inductive coupler 10 generates via electromagnetic induction an oscillating magnetic field from the oscillating current signal. The oscillating magnetic field provides a mechanism for wirelessly transferring power from the transmitter 4 to the receiver 6. The transmitter 4 is not electrically coupled to the receiver 6.

The receiver 6 comprises a secondary inductive coupler (or receiver antenna) 12 which is electrically coupled with powered circuity 14. In operation, the oscillating magnetic field generated by the primary inductive coupler 10 generates via electromagnetic induction an oscillating voltage signal in the secondary inductive coupler 12. The oscillating voltage signal is then used to drive the powered circuitry 14. The powered circuitry 14 could include any type of electric load, component, device or appliance which can be powered from the secondary inductive coupler 12. For example, the powered circuitry 14 may include a rectifier to convert an oscillating (or alternating) current generated from the induced oscillating voltage signal into a direct current or DC signal. For example, some electric loads may require a DC input signal rather than an oscillating or alternating (AC) input. Further, the powered circuitry 14 may further include any of the following example components or devices: a heating element, a communications modules (e.g. a wireless communications module such as a Bluetooth module or a WiFi module), an imaging apparatus (e.g. a camera), an apparatus for generating and delivering electromagnetic energy (e.g. RF and/or microwave energy) for treating (e.g. ablating or coagulating) biological tissue. As such, the system 2 may find application in various fields, including but not limited to medicine (e.g. electrosurgery and/or internal patient monitoring), robotics, and mobile computing (e.g. wireless charging of mobile computing devices)

According to the above, the system 2 is able to power the powered circuity 14 from the power signal source 8 without there being a wired connection therebetween.

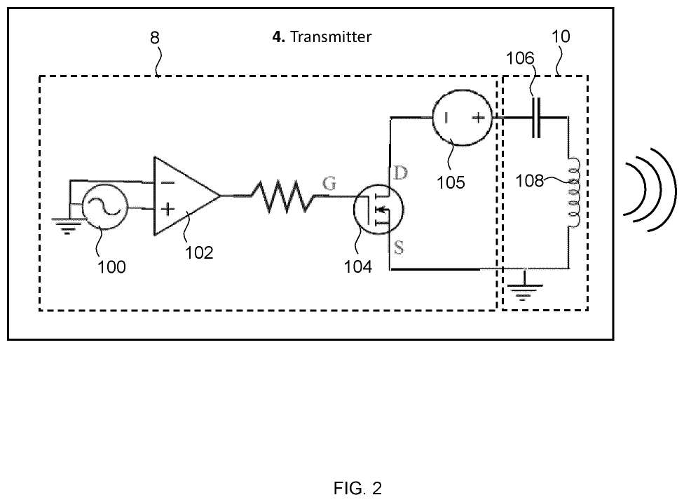

FIG. 2 provides an example implementation of the power signal source 8 and the primary inductive coupler 10.

As seen in FIG. 2, an oscillator 100 provides an oscillating control signal to an amplifier 102. The oscillating control signal may be an oscillating voltage signal having a frequency in the MHz range (e.g. 9.9 MHz). The amplifier 102 amplifies this oscillating control signal to form an oscillating drive signal which has the same frequency as the oscillating control signal but is more powerful such that the oscillating drive signal possesses enough power to drive a MOSFET 104. Specifically, the MOSFET 104 is a voltage controlled current source and, therefore, generates an oscillating current signal (using current supply 105) based on the oscillating drive signal. The oscillating current signal has the same frequency as the control signal and drive signal. This oscillating current signal is then provided to the primary inductive coupler 10. As described above, the primary inductive coupler 10 uses the oscillating current signal to generate an oscillating magnetic field via electromagnetic induction.

The primary inductive coupler 10 comprises a series inductor-capacitor (LC) circuit having capacitor 106 and inductor 108. It is to be understood that the inductor 108 comprises a coil of wire. As such, the primary inductive coupler 10 is a resonant circuit. The specific values of the frequency of the oscillator 100, the capacitance of the capacitor 106 and inductance of the inductor 108 are chosen such that resonance occurs. Resonance may be set to occur based on parameters set by the physical geometry of the transmitter and receiver. In this way, the coil of the inductor 108 generates an oscillating magnetic field.

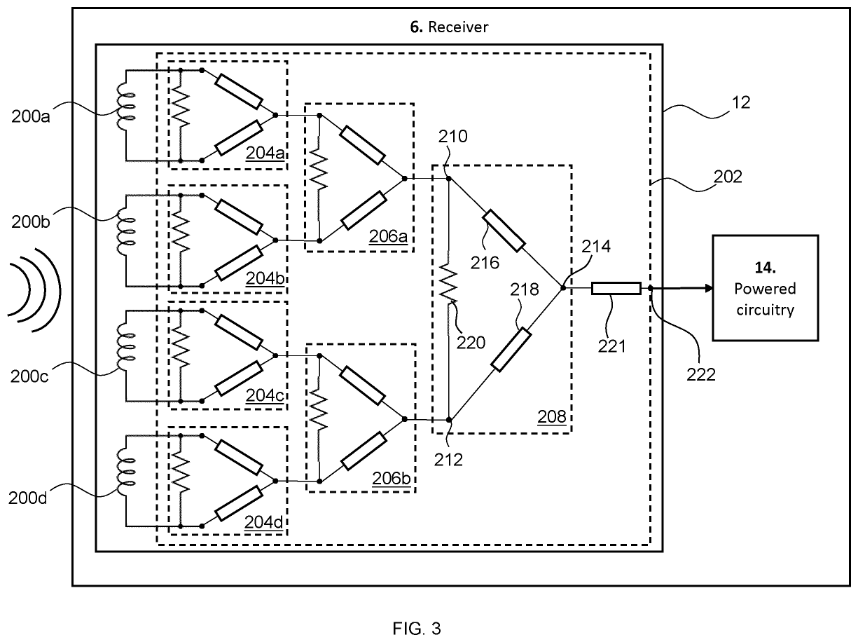

FIG. 3 provides an example implementation of the secondary inductive coupler 12. Specifically, secondary inductive coupler 12 comprises a resonant receiver circuit having a plurality of four coils (aka inductors) 200a-d which are operatively coupled to a combining circuit 202. In an embodiment, the coils 200a-d are made of silver.

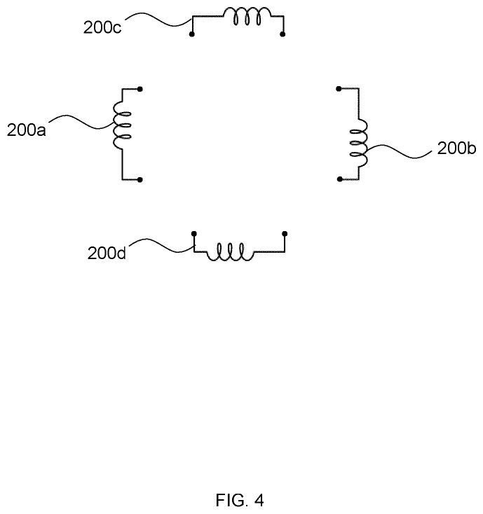

FIG. 3 shows the electric connections between the coils 200a-d and the combining circuit 202. However, it is to be understood that the physical arrangement of the coils 200a-d may be such that at least some of the coils 200a-d are physically orientated at a different angle to at least some of the other coils. For example, in an embodiment, each coil 200a-d may be orientated at a different angle to each other coil. FIG. 4 shows such an embodiment in which coil 200c is rotated by 90 degrees compared to coil 200a; coil 200b is rotated by 90 degrees compared to coil 200c, and is rotated by 180 degrees compared to coil 200a; and, coil 200d is rotated by 90 degrees compared to coil 200b, is rotated by 180 degrees compared to coil 200c, and is rotated by 270 degrees compared to coil 200a. As such, each coil is at a different orientation angle to each other coil. For clarity, the combining circuit 202 has been omitted in FIG. 4; however, it is to be understood that the combining circuit 202 would be connected to the coils 200a-d as shown in FIG. 3. Further, whilst the coils 200a-d are shown with particular unique angles in FIG. 4, it is to be understood that in some other embodiments each coil 200a-d may have a different unique angle to those shown. Also, whilst in FIG. 4 the coils 200a-d are uniformly angularly orientated (i.e. with 90 degrees between each adjacent coil), in some other embodiments, the coils a-d may be irregularly or non-uniformly angularly orientated.

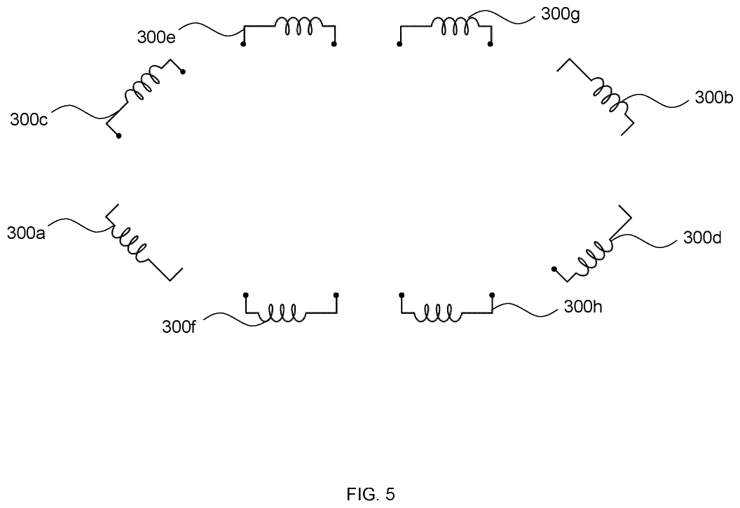

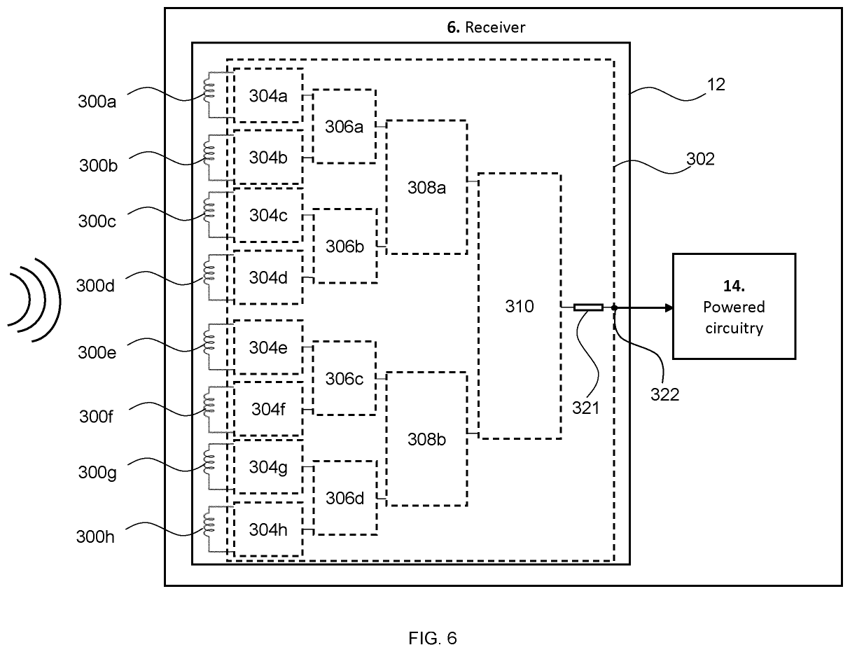

FIG. 5 shows an alternative embodiment to FIG. 4, having a plurality of eight coils 300a-h. In an embodiment, the coils 300a-h are made of silver. In FIG. 5, at least some of the coils 300a-h are physically orientated at the same angle to other coils. Specifically, the coils 300a, 300c, 300b, 300d are each orientated at a unique angle. However, the coils 300e and 300g are orientated at the same angle to each other, and the coils 300f and 300h are orientated at the same angle to each other. As was the case with FIG. 4, for clarity, the combining circuit has been omitted in FIG. 5; however, it is to be understood that the combining circuit would be connected to the coils 300a-h as shown in FIG. 6, which is described in detail below.

For optimal power transfer from a primary coil (e.g. inductor coil 108) to a secondary coil (e.g. any one of coils 200a-d, or 300a-h) the two coils should be parallel to each other. As one coil rotates relative to the other coil from a parallel configuration the amount of power transferred reduces. When the two coils are perpendicular to each other there is no power transfer between the coils. As such, the relative orientation between the primary and secondary coils affects the amount of power transmitted from the primary coil to the secondary coil, wherein power transmission is best when the two coils are parallel to each other and worst when the two coils are perpendicular to each other.

If the receiver 6 is moveable or rotatable relative to the transmitter 4, the relative angle or orientation between the primary and secondary coils can vary. When the primary and secondary coils are parallel to each other, or close to parallel, power transfer will be good, but when the primary and secondary coils are perpendicular to each other, or close to perpendicular, power transfer will be poor. Such variability can be problematic where the powered circuitry 14 requires a constant, or near constant, supply of power to operate correctly. Therefore, having coils at multiple different relative angles or orientations can smooth out power transfer to make it more consistent as the relative angle or orientation between the transmitter 4 and receiver 6 varies. For example, when a first angle exists between the transmitter 4 and the receiver 6, the coil 108 may be parallel to coil 200a but perpendicular to coil 200c. As such, the powered circuity 14 may receive power from the induced power obtained by coil 200a (e.g. at an optimal level), but coil 200c may not contribute any power to the powered circuity 14. As the receiver 6 moves relative to the transmitter 4, the coil 200a may become less parallel and more perpendicular with respect to the coil 108, and the coil 200c may become more parallel and less perpendicular with respect to the coil 108. As this happens, both coils 200a and 200c may provide power to the powered circuitry 14, possibly at a sub-optimal level because neither secondary coil is parallel to the primary coil. Further, as the receiver 6 continues to move relative to the transmitter 4, the coil 200a may become perpendicular to the coil 108, and the coil 200c may become parallel to the coil 108. As this happens, the powered circuitry 14 may receive power from the induced power obtained by coil 200c (e.g. at an optimal level), but coil 200a may not contribute any power to the powered circuitry 14.

Therefore, as the receiver 6 moves relative to the transmitter 4, the powered circuity may receive power from the secondary inductive coupler 12 regardless of an angle of orientation between the transmitter 4 and receiver 6. Specifically, since different coils have different orientation angles relative to the transmitter, as one coils moves toward a perpendicular condition, another coil may move away from a perpendicular condition. In this way, certain coils can compensate for other coils to smooth out the overall power collected by the receiver 6.

Additionally, each transmitter and receiver coil combination is configured for optimal power transfer at a specific (i.e. optimal) distance. As the separation between the coils approaches this optimal distance, power transfer efficiency peaks. When this happens the coils are said to be “critically coupled”. Conversely, power transfer efficiency reduces as the separation between the coils moves away from (e.g. becomes bigger or smaller than) the optimal distance. When the two coils are too close, the formation of mutual magnetic flux between the two coils is hindered by the effect of anti-resonance, and power transfer is sub-optimal (e.g. poor)—in this scenario the two coils are said to be “over-coupled”. On the other hand, when the coils are too far apart, most of the magnetic flux from the primary coil misses the secondary coil, and power transfer efficiency is again sub-optimal (i.e. poor)—in this scenario the two coils are said to be “loosely coupled”. The optimal distance depends on a coupling coefficient of the transmitter and receiver coils. The coupling coefficient of a coil depends on various attributes of the coil, including: a coil inductance, a number of turns in the coil, a permeability of the material (e.g. wire) used to form the coil, a cross-sectional area of the material (e.g. wire) used to form the coil, a length of the coil, and a skin effect of the material (e.g. wire) used to form the coil.

If the receiver 6 is moveable or rotatable relative to the transmitter 4, the relative distance between the primary and secondary coils can vary. When the primary and secondary coils are separated by their optimal distance, power transfer will be optimal, but when the primary and secondary coils are not separated by their optimal distance, power transfer will be sub-optimal. Also, as the separation distance becomes more different from the optimal distance, power transfer will become more sub-optimal, and eventually negligible or zero. Further, power transfer may be particularly poor when the coils are either over-coupled or loosely coupled. Such variability can be problematic where the powered circuitry 14 requires a constant, or near constant, supply of power to operate correctly. Therefore, having multiple receiver coils configured to couple with the primary coil at multiple different optimal distances can smooth out power transfer to make it more consistent as the relative separation between the transmitter 4 and receiver 6 varies.

In view of the above, the arrangement of FIG. 4 is advantageous because it includes multiple coils positioned at different orientation angles. Therefore, as the receiver 6 varies its orientation angle relative to the transmitter coil, different coils provide power at different orientation angles, such that the receiver 6 can be configured to receive power from the transmitter 4 regardless of an angle of orientation between the transmitter 4 and receiver 6. These advantages are also attainable using the arrangement of FIG. 5. Furthermore, the arrangement of FIG. 5 includes multiple coils configured to provide critical coupling at different distances (e.g. different critical zones) from the transmitter coil. Therefore, as the receiver 6 varies its distance from the transmitter coil, different coils provide power at different separation distances, such that the receiver 6 can be configured to receive power from the transmitter 4 over a wider range of distances than would be achievable with a since receiver coil.

Returning to FIG. 3, as stated above, the coils 200a-d are operatively coupled to the combining circuit 202. The combining circuit 202 will now be described in detail.

The combining circuit 202 comprises a plurality of power combiners 204a-d, 206a-b and 208. Each power combiner functions to combine together two power feeds into a single power feed. The power combiners are arranged into multiple stages: first stage 204a-d, second stage 206a-b and third stage 208. The final stage may be connected to an output 222 of the combining circuit 202 by a single impedance element 221, or the final stage may include the impedance element 221 which is connected in-between the output port of its power combiner (i.e. power combiner 208) and the output 222 of the combining circuit. Each power combiner has the same basic construction including two input ports coupled to an output port.

Taking power combiner 208 as an example, a first input port is labelled 210, a second input port is labelled 212, and the output port is labelled 214. A first impedance element 216 is coupled between the first input port 210 and the output port 214. A second impedance element 218 is coupled between the second input port 212 and the output port 214. A third impedance element 220 is coupled between the first input port 210 and the second input port 212. Each power combiner is a passive device used to combine electromagnetic power received on the first and second input ports 210, 212 into a combined power signal which is provided at the output port 214 for use by another circuit, which in this case is the powered circuitry 14. In an embodiment, each power combiner is a Wilkinson power combiner. The power combiners may be realised in a number of different technologies including coaxial and planar technologies (e.g. stripline or microstrip). However, the embodiment of FIG. 3 may be constructed using microstrip.

Also, each power combiner acts as a lumped element having a characteristic impedance. That is, each power combiner may form a signal adder based on lumped elements, e.g. a combination of series inductors and shunt capacitors. For example, the characteristic impedance is determined by the geometry and materials used to form the power combiner. Specifically, the selected geometry and materials determine values of the impedance elements 216, 218 and 220, and the values of the impedance elements 216, 218 and 220 determine the characteristic impedance of the power combiner. It is to be understood that different power combiners may have different values of impedance elements 216, 218 and 220. Therefore, different power combiners may have different characteristic impedances.

The combining circuit 202 has two main functions.

Firstly, the plurality of power combiners (204a-d, 206a-b and 208) are connected together to combine power received via each of the plurality of coils 200a-d into a combined power signal that is provided to the output 222 of the combining circuit 202. This combined power signal can then be used to power the powered circuitry 14 which is coupled to the output 222.

Secondly, each coil is coupled to the output 222 by a particular combination of power combiners having characteristic impedances which combine together with that coil to form a resonant circuit for receiving power via resonant inductive coupling. For example, coil 200a is coupled to the output 222 by a first combination of power combiners (204a, 206a and 208), whereas coil 200b is coupled to the output 222 by second (i.e. different) combination of power combiners (204b, 206a and 208). Considering coil 200a, the power combiners 204a, 206a and 208 are configured such that their characteristic impedances combine together with coil 200a to form a resonant circuit for receiving power via resonant inductive coupling between primary coil 108 and secondary coil 200a. Additionally, considering coil 200b, the power combiners 204b, 206a and 208 are configured such that their characteristic impedances combine together with coil 200b to form a resonant circuit for receiving power via resonant inductive coupling between primary coil 108 and secondary coil 200b. Stated differently, the combining circuit 202 provides a plurality of signal paths, wherein each signal path connects a different one of the coils 200a-d to the output 222. Also, each signal path contains a plurality of power combiners (e.g. impedance elements), and the power combiners (e.g. impedance elements) on a given path have characteristic impedances which combine into a combined characteristic impedance having a capacitance that resonates with the inductance of the coil connected to that path. For example, considering coil 200a, the power combiners 204a, 206a and 208 have a combined characteristic impedance that is sufficiently capacitance to resonate with the inductance of coil 200a. In an embodiment, each coil is coupled to the output 222 via a unique combination of power combiners. In an embodiment, the impedance element 221 may also form part of each coil's resonant circuit.

Therefore, the combining circuit 202 combines power received by each coil into a single power signal for powering the powered circuity 14. Also, the combining circuit 202 forms a separate resonant circuit with each coil 200a-d so that each coil 200a-d can receive power via resonant inductive coupling.

FIG. 3 illustrates exemplary circuitry of combining circuit 202 for connecting the four coils 200a-d of FIG. 4 to the powered circuitry 14 such that each of the four coils 200a-d can receive power via resonant inductive coupling and such that this received power can be combined together to power the powered circuitry 14. FIG. 6 illustrates exemplary circuitry of a combining circuit 302 for connecting the eight coils 300a-h of FIG. 5 to the powered circuitry 14 such that each of the eight coils 300a-h can receive power via resonant inductive coupling and such that this received power can be combined together to power the powered circuity 14 via an output 322. The combining circuit 302 has the same basic construction as the combining circuit 202. Specifically, the combining circuit 302 comprises a plurality of power combiners 304a-h, 306a-d, 308a-b and 310. Each power combiner 304a-h, 306a-d, 308a-b and 310 has the same basic construction as each of the power combiners 204a-d, 206a-b and 208 described above. The individual components of each power combiner are not shown in FIG. 6 for clarity. Also, the basic functionality of the combining circuit 302 is the same as that of the combining circuit 202, described above. The only difference between the combining circuit 302 and the combining circuit 202 is that the combining circuit 302 has to collect together power from more coils (i.e. eight rather than four), and the combining circuit 302 has to form more resonant circuits (i.e. eight rather than four). Therefore, the combining circuit 302 has additional power combiners compared to the combining circuit 202, i.e. fifteen rather than seven. Also, the combining circuit 302 has an additional fourth stage (i.e. power combiner 310) compared to the combining circuit 202. It is to be understood that where the number of inductors or coils (N) is a square of two, the number of power combiners in the combining circuit will be 2N−1. Also, the number of stages will be 2 to the power of (N−1) or 2^{(N-1)}.

Considering a first stage, the number of first stage power combiners matches the number of coils (e.g. first stage 204a-d has four power combiners to match the four coils 200a-d; and, first stage 304a-h has eight power combiners to match the eight coils 300a-h). Also, each first stage power combiner is associated with a different coil of the plurality of coils, e.g. power combiner 204a is associated with (e.g. connected to) coil 200a, but power combiner 204b is associated with (e.g. connected to) coil 200b. Further, each first stage power combiner has a first input port connected to a first end of its associated coil, and a second input port connected to a second end of its associated coil, e.g. the first input of power combiner 204a is connected to the top end of coil 200a and the second input of the power combiner 204a is connected to the bottom end of coil 200a. This construction is also true of combining circuit 302.

After the first stage, the combining circuit may have one or more further stages, for example, combining circuit 202 has a second stage 206a-b and a third stage 208, whereas the combining circuit 302 has a second stage 306a-d, a third stage 308a-b and a fourth stage 310. Considering an example further stage (e.g. the second stage 306a-d), the number of power combiners in that further stage (i.e. four) matches half the number of power combiners in an adjacent previous stage (that is, the first stage has eight, so the second stage has half of this, i.e. four). Also, each power combiner in that further stage (i.e. each of 306a-d) is associated with a different pair of power combiners from the adjacent previous stage (i.e. the first stage), and each power combiner from the adjacent previous stage (i.e. 304a-h) is only associated with a single power combiner from that further stage (i.e. the second stage). For example, second stage power combiner 306b is associated with (e.g. connected to) first stage power combiners 304c-d, and first stage power combiners 304c-d are only connected to second stage power combiner 306b. Further, each power combiner in that further stage (i.e. each of 306a-d) has a first input port connected to the output port of one of its associated pair of power combiners from the adjacent previous stage (i.e. 306b has a first input connected to the output of 304c), and a second input port connected to the output port of the other of its associated pair of power combiners from the adjacent previous stage (i.e. 306b has a second input connected to the output of 304d). This construction is also true of combining circuit 202.

It is noted that the order in which the coils are connected in the combining circuit 302 can be specifically selected to improve power transfer and/or power collection. Specifically, as seen in FIG. 6, the outputs from coils 300a and 300b are both connected to the power combiner 306a. Looking at FIG. 5, it can be seen that coils 300a and 300b are orientated roughly opposite to each other. The same can be seen for all the other coils 300b-h, that is: 300c and 300d have opposite orientations and are both coupled to power combiner 306b; 300e and 300f have opposite orientations and are both coupled to power combiner 306c; and, 300g and 300h have opposite orientations and are both coupled to power combiner 306d. This arrangement is chosen to improve power transfer and collection, for example, the total power transferred to the coils 300a-h and/or the total power collected at the output 322. Specifically, this arrangement balances the amount of power on adjacent paths from the coils 300a-h to the output 322. That is, in order to improve balance in the system, and thereby improve total power transfer and collection, it is preferable that adjacent paths of the combining circuit 302 carry amounts of power which are as similar as possible. For example, looking at FIG. 6, it is preferable that the power on first input port of the power combiner 306a is most similar to the power on the second input port of the power combiner 306a. This is achieved by pairing together two coils which generate the most similar amount of average power in a given situation. For example, two coils which are configured for optimal power transfer at similar orientations and/or at similar distances generate a similar amount of average power in a given situation. Conversely, two coils which are configured for optimal power transfer at very different orientations and/or distances generate different amounts of average power in a given situation. Therefore, the coils 300a-h are arranged in the combining circuit 302 to improve balance and power transfer. Specifically, the following pairs of coils are established in the second stage: 300a:300b, 300c:300d, 300e:300f, and 300g:300h. Further, the same strategy is adopted in each subsequent stage. For instance, it is established that the pair 300a:300b produces a similar amount of average power in a given situation to the pair 300c:300d and so these pairs are both input to the same power combiner 308a. Also, it is established that the pair 300e:300f produces a similar amount of average power in a given situation to the pair 300g:300h and so these pairs are both input to the same power combiner 308b. If further stages were present in the combining circuit, then this assessment would be performed for each stage (other than the final stage which includes only one power combiner). It is to be understood that the average power generated by different coil and power divider combinations in a given situation can be determined empirically, and the results can be used to choose or select the connections between the power combiners or power combiner stages.

In summary, therefore, the connections between the plurality of coils (300a-h) and the combining circuit (302), and the connections between the plurality of power combiners (304a-h, 306a-d, 308a-b, 310) in the combining circuit (302) are selected to minimise differences between the two power signals input to each power combiner. This is achieved by pairing together coils which provide the most similar average power in a given situation (e.g. a test situation). Also, this is achieved by pairing together power combiners of the same stage which provide the most similar average power in the given situation. It is noted that the connections between the coils 200a-d and the power combiners 204a-d, 206a-b and 208 of the combining circuit 202 are established in the same manner.

Based on the above, whilst examples of receivers having four and eight coils have been provided, in some other embodiments, the receiver 6 could have more or less that these numbers of coils, e.g. more than eight or less than four. Furthermore, it is clear from the above description how to modify the combining circuit as the number of coils changes. It is noted that as the combining circuit is adapted to the number of coils, the combining circuit functions to combine power received via each coil into a single power signal for powering the powered circuity 14. Also, the combining circuit forms a resonant circuit with each coil so that each coil can receive power via resonant inductive coupling.

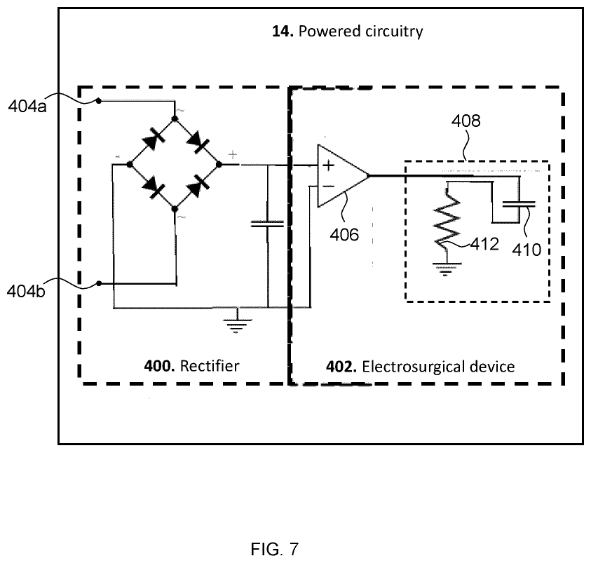

FIG. 7 illustrates an example implementation of the powered circuitry 14, which is described in detail below. However, it is to be understood that the implementation of FIG. 7 is merely one possible example of the powered circuity 14. In some other embodiments, the powered circuitry 14 can include other electronic devices, appliances, or circuits for transforming electrical energy into other forms. For example, the powered circuitry 14 may transform the electrical energy into a different type of electrical energy, such as, for example, electrical energy which represents a physical property (e.g. temperature or pressure) or which represents data of some kind (e.g. communication data). Additionally or alternatively, the powered circuitry 14 may transform the electrical energy into a different type of energy, such as, thermal energy (for heating or cooling), electromagnetic energy (e.g. gamma rays, x-rays, UV light, visible light, IR light, RF signals, microwave signals), and/or sound energy (e.g. ultrasonic vibrations, audible vibrations).

Turning to FIG. 7, the powered circuitry 14 comprises a rectifier 400 operably coupled to an electrosurgical device 402. The rectifier 400 has input terminals 404a and 404b which are coupled (not shown) to the secondary inductive coupler 12 so as to receive power therefrom. For example, the rectifier 400 may be coupled to the output 222 of the combining circuit 202 or the output 322 of the combining circuit 302. As shown in FIG. 7, the rectifier 400 may be a full wave bridge rectifier; however, it is to be understood that in some other embodiments the rectifier 400 may be a different type of rectifier, such as, for example, a half-wave rectifier, or a centre tapped rectifier. In any case, the rectifier 400 functions to convert an alternating current signal provided by the secondary inductive coupler 12 into a direct current or DC signal.

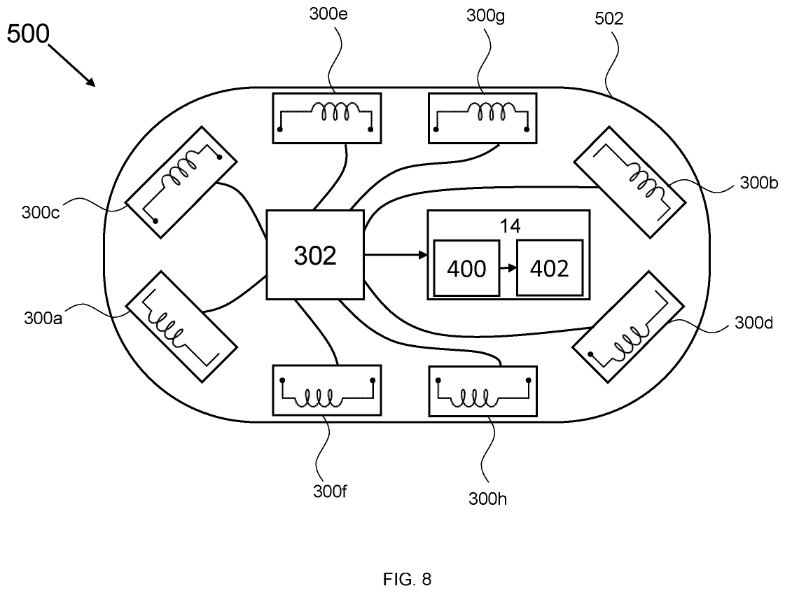

The electrosurgical device 402 receives the rectified power signal from the rectifier 400 and uses it to generate and radiate electromagnetic energy, such as, non-ionising RF or microwave energy. Specifically, in the embodiment of FIG. 7, the receiver 6 is part of a capsule 500, as shown in FIG. 8. The capsule 500 comprises a housing 502 which encases or contains the receiver 6. It is to be understood that the capsule is a medical device which is intended to be inserted or ingested (e.g. swallowed) by a patient. For example, the capsule 500 may be an endoscopic capsule. As such, the capsule 500 is sized and shaped to facilitate being swallowed. For instance, a shape of the housing 502 is substantially sphero-cylindrical, i.e. shaped like a pill. Also, a maximum length of the housing 502 may be about 20 mm±5 mm, and a maximum width of the housing 502 may be about 10 mm±5 mm. Additionally, the housing may be formed from a biocompatible material (e.g. Parylene C or PTFE). Alternatively, a layer of biocompatible material may be applied to an outer surface of the housing 502. The biocompatible layer may have a thickness of 10 μm or less.

As an illustration, the coils 300a-h are shown in a possible position within the housing 502. It is however to be understood that the capsule 500 is not limited to being used with particular number of coils. Also, in some other embodiments, the coils may be positioned differently, for example, with different relative orientations. As explained above with reference to FIG. 6, the coils 300a-h are connected to the combining circuit 302, and the coils 300a-h, with the combining circuit 302 receive power via resonant inductive coupling and output a combined power signal to the powered circuitry 14. This combined power signal is rectified by the rectifier 400 to provide a DC signal which is provided to the electrosurgical device 402, as described above.

Returning to FIG. 7, the electrosurgical device 402 comprises a microwave power amplifier 406 which is coupled to the rectifier 400 for generating microwave electromagnetic energy from the rectifier's DC output signal. Also, the electrosurgical device 402 comprises a microwave transmission line 408 (represented by capacitance 410 and series connected resistor 412) which is coupled to the microwave power amplifier 406 for receiving and radiating the microwave electromagnetic energy into biological tissue surrounding the capsule 500. That is, when the capsule 500 is swallowed by a patient, it enters inside the patient's body. The capsule 500 may actively or passively travel to a treatment zone or site in the body (e.g. in the gastrointestinal tract) at which microwave energy is to be radiated to treat tissue at the treatment site, for example, to coagulate or ablate the tissue. In an embodiment, the capsule 500 is actively steered to the treatment site via a magnetic steering apparatus which is located outside the patient's body. As such, the capsule 500 may include a ferrous or magnet element (not shown) which reacts with (e.g. is attracted to and repelled by) the magnetic steering apparatus so that the path of travel of the capsule 500 through the patient to the treatment site can be guided or controlled from via the magnetic steering apparatus from outside the patient's body.

In an embodiment, the electrosurgical device 402 is configured such that an impedance of the transmission line 408 is matched to an impedance of the type of biological tissue to be treated by the device (aka target tissue type) in order to ensure even (or uniform) energy delivery into the tissue. For example, it is known to construct an equivalent electrical circuit for biological tissue. Specifically, in this equivalent electric circuit, a resister Rᵢ, is connected in series with a capacitor Cₘ, and then Rᵢ, and Cₘ are both connected in parallel with a resistor Rₑ. Rₑ represents extracellular resistance, Rᵢ represents intracellular resistance, and Cₘ represents electrical capacitance of the cell membrane. Rₑ, Rᵢ, and Cₘ are the resistance component derived from extracellular fluid, resistance component derived from intracellular fluid, and capacity component derived from the cytoplasmic membranes (overall, combined cells to make tissue, dielectric property of biological material of which the electrical field crosses), respectively. Also, Rₑ, Rᵢ, and Cₘ vary between different tissue types and, as such, a target tissue type has associated values of Rₑ, Rᵢ, and Cₘ and, therefore, an associated tissue impedance. The geometry of the transmission line 408 can be selected (e.g. set) to provide an impedance (e.g. values of capacitance 410 and resistance 412) which matches or is similar to the impedance of the target tissue type. In this way, the electrosurgical device evenly (or uniformly) delivers energy into the target tissue at the treatment site.

In an embodiment, the microwave energy delivered by the electrosurgical device 402 may be used to treat trauma bleeds, for example, by coagulating tissue at the treatment site. Additionally or alternatively, the microwave energy may be used to treat lesions or tumours, for example, by ablating tissue at the treatment site. Also, it is to be understood that whilst the embodiment of FIG. 7 includes an electrosurgical device for generating microwave energy via a microwave power amplifier, in some other embodiments a different type of high-frequency electromagnetic energy may be generated, for example, RF energy may be generated by an RF power amplifier.

In an embodiment, rather than the capsule being swallowed by a patient and used to perform operations in the gastrointestinal tract, the capsule 500 may instead be inserted into a vascular system, for example, the femoral artery. In this case, procedures may have to be quicker to avoid blocking blood flow in the vessel. Also, any coagulation performed would need to be restricted to the vessel itself so as not to clod the blood conveyed by the vessel.

The above-described embodiments of the receiver 6 are particularly well suited to powering a capsule to be ingested by or inserted into a patient, such as the capsule 500, which may be an endoscopic capsule. Specifically, as the capsule 500 travels through the patient's body, and once the capsule 500 has arrived at the treatment site, it can be hard to control the relative angle or orientation between the primary coil 108 in the transmitter 4 and a single secondary coil (e.g. coil 300a) in the receiver 6. Also, it can be hard to control the relative spacing between the primary coil 108 and the secondary coil 300a. It is noted that even though a magnetic steering apparatus may be used to guide the capsule through the patient to the treatment site, the exact orientation of the capsule and the exact spacing between the capsule and the primary coil 108 can be hard to control. Therefore, an advantage of the above-described embodiments, is that multiple secondary coils are provided (e.g. coils a-h) in the receiver 6, wherein different coils enable optimal power transfer at different relative angles between the capsule 500 and the primary coil 108. Also, different coils enable optimal power transfer at different distances between the capsule 500 and the primary coil 108. In this way, it is possible to develop a capsule wherein power transfer to the capsule is insensitive to relative orientation and spacing between the capsule and the primary coil 108. For example, at a particular relative orientation and spacing between the capsule 500 and the primary coil 108, one or more of the coils 300a-h may be experiencing optimal power transfer, one or more of the coils 300a-h may be experiencing no power transfer, and one or more of the coils 300a-h may be experiencing sub-optimal power transfer. However, all the coils 300a-h are coupled to the combining circuit 302 such that whatever power is received via the different coils 300a-h, the received power is combined and provided to the powered circuitry 14 on-board the capsule. In this way, embodiments provide an improved mechanism for powering an ingestible/insertable capsule (or endoscopic capsule).

In some other embodiments, the powered circuitry of the capsule may additionally or alternatively include an imaging device (e.g. a camera) for inspecting and monitoring internal structures (e.g. vascular structures) of a patient. For example, the imaging device may be configured to capture images in timed intervals (e.g. twice per second). In this case, the housing of the capsule may include a window portion which is transparent such that the imaging device can see through the housing. Also, a light source may be included to illuminate the tissue surrounding the capsule or window each time the imaging device captures a new image. Additionally or alternatively, the powered circuitry may include one or more biosensors for detecting the presence or concentration of a biological analyte, such as a biomolecule, a biological structure or a microorganism. In this case, the housing of the capsule may have an aperture which allows at least part of the biosensor to contact tissue in treatment site. Additionally or alternatively, the powered circuitry may include a thermal module (e.g. heating element) for changing a temperature of tissue at the treatment site. For example, the thermal module may be used to heat tissue (e.g. cancerous tissue) at the treatment site to activate heat activated drugs, such as, heat activated chemotherapy drugs.

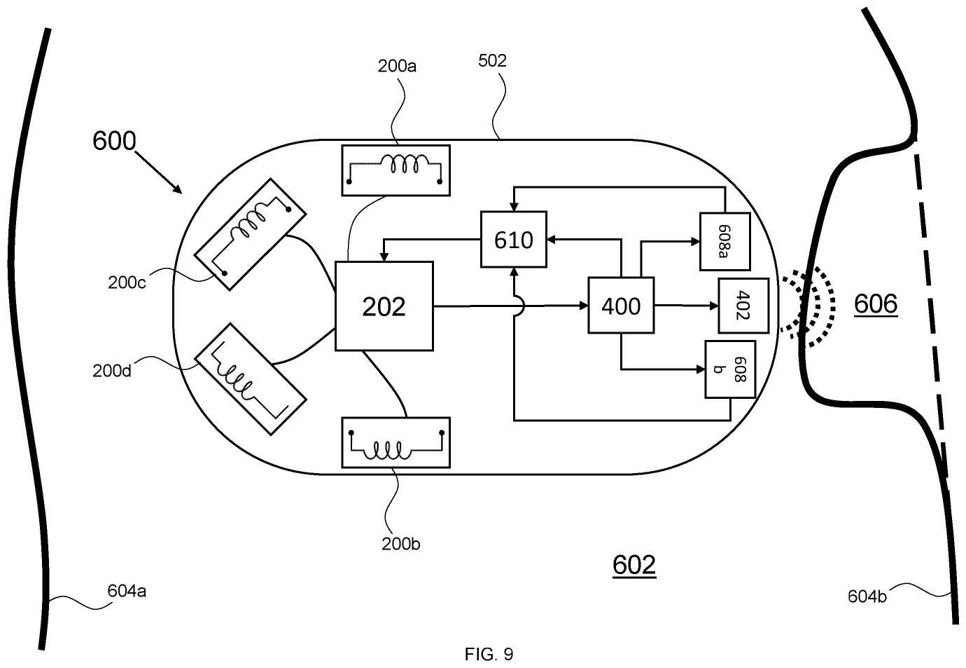

FIG. 9 shows a capsule 600, which is a variant of the capsule 500 of FIG. 8. The capsule 600 includes the above-described structure and functionality of the capsule 500, but has the following differences.

Where the capsule 500 includes eight coils 300a-h connected to the combining circuit 302, the capsule 600 includes four coils 200a-d connected to the combining circuit 202. That said, the capsule 600 can include any number of coils and a combining circuit which is adapted to that number of coils. Also, the relative orientation of each coil, and the critical zone of each coil, can vary between embodiments so that power can be received by the receiver 6 regardless of the orientation between the transmitter 4 and receiver 6, and so that power can be received by the receiver 6 within a wide range of separation distances between the transmitter 4 and receiver 6.

The capsule 600 includes the rectifier 400 for generating a DC power signal from the power received via resonant inductive coupling. Also, the electrosurgical device 402 receives the rectified power signal from the rectifier 400 and uses it to generate and radiate electromagnetic energy, such as, non-ionising RF or microwave energy. FIG. 9 illustrates the capsule 600 inside a lumen 602 of a patient's digestive tract (which is shown in cross-section in FIG. 9). The epithelium of the digestive tract is represented by lines 604a and 604b. The epithelium 604b includes a tumor 606, which represents the treatment site or zone mentioned above. Dotted lines in FIG. 9 illustrate the radiation of electromagnetic energy from the electromagnetic device 402 to the tumor 606 in the treatment site. The electromagnetic energy may be used to ablate and/or coagulate tissue at the treatment site in order to kill the tumor 606.

As stated above with respect to the capsule 500, the capsule 600 may be guided to the treatment site via a magnetic steering apparatus. However, it may be difficult to confirm when the capsule 500/600 is in position. Moreover, if the capsule 500/600 is not in position, there is a risk that the electromagnetic energy may be radiated into healthy tissue rather than unhealthy tissue (e.g. a tumor). Therefore, the capsule 600 includes one or more sensors which generate electrical signals corresponding to the capsule's surroundings (e.g. the tissue surrounding the electromagnetic device 402). FIG. 9 shows that the capsule 600 includes two sensors 608a and 608b, but it is to be understood that in some other embodiments there could be more or less than two sensors. Also, the exact position of the sensors within the housing 502 may vary between embodiments, for example, the electromagnetic device 402 could be located at one end of the capsule 600 and the one or more sensors 608a, 608b could be located at an opposite end of the capsule 600. In any case, each sensor 608a, 608b may be an imaging module which generates an electric signal based on electromagnetic signals (e.g. infrared signals, ultraviolet, visible light) received (e.g. reflected) from the treatment site. For example, each sensor may be a Fujikura 40K CMOS Image Sensor Module. Furthermore, each sensor receives power from (i.e. is powered by) the rectifier 400. As such, each sensor is powered from power received by the capsule 600 via resonant inductive coupling.

Each sensor 608a, 608b has a sensor output port from which is output the electrical signal (e.g. voltage signal) that corresponds to (i.e. provides a representation of) the capsule's current surroundings. The sensor output of each sensor 608a, 608b is connected to a signal conditioning unit 610. The signal conditioning unit 610 is also connected to the rectifier 400 so as to receive power therefrom. The signal conditioning unit 610 conditions the electrical signal output from each sensor 608a, 608b so that it is suitable for transmission from the receiver 6 via the combining circuit 202 and coils 200a-d. That is, the combining circuit 202 and coils 200a-d form a resonant transmitter circuit configured to transmit the conditioned electrical signals to the transmitter 4 via resonant inductive coupling. Specifically, the signal conditioning unit 610 amplifies the electrical signals from the sensors 608a, 608b so that they are powerful enough to be transmitted via resonant inductive coupling and received at the transmitter 4. For example, the signal conditioning unit 610 may include a power amplifier which amplifies a voltage of the electrical signals output from the sensors. Additionally, the signal conditioning unit 610 changes (e.g. increases or decreases) a frequency of the electrical signals output from the sensors to reduce or avoid interference between the sensor signals transmitted from the receiver 6 to the transmitter 4 and the power signals transmitted from the transmitter 4 to the receiver 6. For example, the signal conditioning unit 610 may include a frequency divider to reduce the frequency of the sensor signals and/or a frequency multiplier to increase the frequency of the sensor signals. It is to be understood that regardless of whether the sensor signal frequency is increased or decreased, the conditioned sensor signals have a frequency which cooperates with the frequency of the power signals, wherein frequencies “cooperate” if interference (constructive or destructive) is avoided or reduced. In an embodiment, the power signals may be about 9 MHz and the conditioned sensor signals may be about 1 MHz.

Therefore, since sensors 608a and 608b are positioned either side of the electrosurgical device 402, the signals from sensors 608a and 608b provide an accurate representation of the physical environment (e.g. the tissue) in front of the electrosurgical device 402. Accordingly, a user can receive these representations at the transmitter 4 to confirm when the capsule 600 is at the treatment site (e.g. at the tumor 606). For example, the conditioned sensor signals may be used to generate an image on a display device (e.g. monitor) connected to the transmitter 4. A human operator can then use the image to determine when the capsule 600 is in position and, when it is, the user can activate the electrosurgical device 402 to deliver electromagnetic energy into the treatment site for tissue treatment. Specifically, the electromagnetic energy may be microwave energy which ablates and/or coagulates tumor 606. Activation of the electrosurgical device may be via a specific control signal which is incorporated in the power signal transmitted from the transmitter to the receiver.

In the above described embodiments, the combining circuits include power combiners having only two input ports and a single output port. However, in at least some other embodiments, different power combiner structures may be used. For example, each power combiner may have more than two input ports, for example, three, four, five or more. In any case, each power combiner functions to combine together the signals received on each input port into a combined signal which is output from the output port of the power combiner. In this case, as in the embodiments explained above, the power combiners of the combining circuit are connected together to combine power received from each of the receiver coils into a combined power signal that is provided to an output of the combining circuit. Also, each receiver coil is coupled to the output of the combining circuit by a combination of power combiners (or impedance elements), and this combination of power combiners (or impedance elements) has a characteristic impedance which combines together with that coil to form a resonant circuit for receiving power via resonant inductive coupling. As before, each power combiner may have a characteristic impedance, for example, each power combiner may act as a lumped element having a particular characteristic impedance. That is, each power combiner may form a signal adder based on lumped elements, e.g. a combination of series inductors and shunt capacitors.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1. A receiver for wirelessly receiving power from a transmitter, the receiver comprising a resonant receiver circuit having a plurality of coils operatively coupled to a combining circuit,
wherein each coil, with the combining circuit, is arranged to receive power via resonant inductive coupling, and
wherein the combining circuit is arranged to combine power received from the plurality of coils for provision to an electric load.

2. The receiver of claim 1, wherein at least two coils of the plurality of coils are orientated at different angles to each other.

3. The receiver of claim 1 or 2, wherein at least two coils of the plurality of coils are configured for critical coupling at different distances to each other.

4. The receiver of any preceding claim, wherein the combining circuit comprises a plurality of impedance elements, wherein the plurality of impedance elements are connected together to combine power received from each of the plurality of coils into a combined power signal that is provided to an output of the combining circuit, and
wherein each coil is coupled to the output of the combining circuit by a combination of impedance elements, the combination of impedance elements having a characteristic impedance which combines together with that coil to form a resonant circuit for receiving power via resonant inductive coupling.

5. The receiver of any preceding claim, wherein the combining circuit comprises a plurality of power combiners, each power combiner having two input ports coupled to an output port and being operable to provide at the output port a combination of separate power signals received at both input ports, and
wherein the plurality of power combiners are connected together to combine power received from each of the plurality of coils into a combined power signal that is provided to an output of the combining circuit, and
wherein each power combiner has a characteristic impedance, and wherein each coil is coupled to the output of the combining circuit by a combination of power combiners having characteristic impedances which combine together with that coil to form a resonant circuit for receiving power via resonant inductive coupling.

6. The receiver of claim 5, wherein the plurality of power combiners are grouped into multiple stages including a first stage, wherein the number of first stage power combiners matches the number of coils in the plurality of coils, and each first stage power combiner is associated with a different coil of the plurality of coils, and
wherein each first stage power combiner has a first input port connected to a first end of its associated coil, and a second input port connected to a second end of its associated coil.

7. The receiver of claim 6, wherein the multiple stages include one or more further stages, and wherein, for each further stage, the number of power combiners in that further stage matches half the number of power combiners in an adjacent previous stage, and each power combiner in that further stage is associated with a different pair of power combiners from the adjacent previous stage, and each power combiner from the adjacent previous stage is only associated with a single power combiner from that further stage, and each power combiner in that further stage has a first input port connected to the output port of one of its associated pair of power combiners from the adjacent previous stage, and a second input port connected to the output port of the other of its associated pair of power combiners from the adjacent previous stage.

8. The receiver of any one of claims 5 to 7, wherein connections between the plurality of power combiners in the combining circuit are selected to minimise differences between the power signals provided at the first and second input ports of each power combiner.

9. The receiver of claim 8, when dependent on claim 7, wherein the power combiners from the adjacent previous stage are paired together based their average power output.

10. The receiver of any one of claims 5 to 9, wherein at least one of the power combiners is a Wilkinson power combiner.

11. The receiver of any one of claims 5 to 10, wherein at least one of the power combiners is formed from a microstrip electrical transmission line.

12. The receiver of any preceding claim, further comprising an electric load coupled to the combining circuit to receive power therefrom.

13. The receiver of claim 12, wherein the electric load comprises a rectifier to convert the power received from the combining circuit into a direct current (DC) signal.

14. The receiver of claim 13, wherein the electric load comprises an electrosurgical apparatus for generating and delivering electromagnetic energy into a treatment site around the receiver for treating biological tissue.

15. The receiver of claim 14, wherein the electrosurgical apparatus comprises:
a microwave power amplifier coupled to the rectifier for generating microwave electromagnetic energy from the DC signal, and
a transmission line coupled to the microwave power amplifier for delivering the microwave electromagnetic energy into biological tissue in the treatment site.

16. The receiver of claim 15, wherein the transmission line is arranged to have an impedance that matches an impedance of a target biological tissue in the treatment site.

17. The receiver of any one of claims 13 to 16, wherein the electric load comprises a sensor for generating an electrical signal based on an environment of the receiver, the sensor being operatively coupled to the rectifier so as to be powered by the DC signal, the sensor being operatively coupled to the combining circuit so as to provide the electrical signal thereto, and wherein each coil, with the combining circuit, provides a resonant transmitter circuit arranged to transmit the electrical signal via resonant inductive coupling.

18. The receiver of claim 17, wherein the electric load comprises a signal conditioning unit operatively coupled in-between the sensor and the combining circuit, the signal conditioning unit being operable to vary a characteristic of the electrical signal before transmission.

19. A capsule for ingestion by a patient, the capsule comprising a housing containing a receiver according to any preceding claim.

20. The capsule of claim 19, wherein a shape of the housing is substantially sphero-cylindrical, and wherein the plurality of coils are arranged in a substantially oval shape which follows an inside surface of the housing.

21. A wireless power transfer system comprising:
a transmitter for wirelessly transmitting power, the transmitter comprising a resonant transmitter circuit having a coil arranged to transmit power wirelessly via resonant inductive coupling, and
a receiver according to any one of claims 1 to 18, or a capsule of claim 19 or 20, for wirelessly receiving power from the transmitter.
